(19) United States
(12) Patent Application Publication
(10) Pub. No.: US 2008/0279805 A1
(43) Pub. Date: Nov. 13, 2008

(54) USE OF NON-FILM-FORMING CATIONIC LATICES FOR INCREASING THE VOLUME OF THE HAIR

(75) Inventor: Franck GIROUD, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL S.A.

(21) Appl. No.: 12/175,707

(22) Filed: Jul. 18, 2008

Related U.S. Application Data
(63) Continuation of application No. 10/678,144, filed on Oct. 6, 2003, now abandoned.
(60) Provisional application No. 60/434,660, filed on Dec. 20, 2002.

(30) Foreign Application Priority Data
Oct. 4, 2002 (FR) 02 12361

(51) Int. Cl. A61K 8/72; A61K 8/81; A61Q 5/00

(52) U.S. Cl. 424/70.16; 424/70.11; 424/70.17; 132/202

(57) ABSTRACT

A hair treatment composition comprising, a dispersion, in a cosmetically acceptable liquid medium, of non-film-forming cationic latex particles
formed from at least one synthetic polymer, obtained by free-radical polymerization, with a glass transition temperature (T_{g}) of greater than or equal to 30° C.,
with a mean particle size ranging from 10 nm to 200 nm, and
with a zeta surface potential (ζ) of greater than +20 mV;
   and also the use of such a composition for increasing the volume of the hair.

This application claims benefit of U.S. Provisional Application No. 60/434,660, filed Dec. 20, 2002.

Disclosed herein are the use of fine, non-film-forming cationic latex particles to increase the volume of the hair, hair compositions comprising such non-film-forming cationic latex particles and a hair treatment process using these compositions.

There currently exist on the market a certain number of products based on film-forming polymers that can give the hair volume. However, the deposit of such film-forming polymers on the surface of the hair may give it an unnatural feel.

Another well-known approach for increasing the volume of the hairstyle is the permanent reshaping of the hair via a process successively using a reducing agent and an oxidizing agent. However, exposure of the keratin fibers to such relatively aggressive physicochemical conditions can lead to degradation of the keratin and impairment of the mechanical and cosmetic properties of the hair. Moreover, this type of process also can modify the level of curliness of the hair, an effect not always desired by people seeking to increase the volume of their hair.

Thus, apparently no treatment currently exists for increasing the volume of the hair without changing the shape of the hair or impairing its feel.

The present inventor has discovered that it may be possible to give the hair volume, without degradation of the keratin fibers or an unpleasant change in the feel of the hair, by treating the hair with a composition comprising non-film-forming cationic latex particles as described below. As a result of their very small size and their cationic charge, the latex particles can bind to the keratin fibers and can remain adsorbed thereon without, however, forming a polymer film liable to change the feel of the treated hair.

Disclosed herein are hair treatment compositions comprising a dispersion, in a cosmetically acceptable liquid medium, of non-film-forming cationic latex particles

formed from at least one synthetic polymer, obtained by free-radical polymerization, with a glass transition temperature (T_{g}) of greater than or equal to 30° C.,

with a mean particle size ranging from 10 nm to 200 nm, and

with a zeta surface potential (ζ) of greater than +20 mV,

and also disclosed herein is the use of such compositions for increasing the volume of the hair.

As used herein, the term “latex” means a suspension of particles made of at least one synthetic polymer, obtained by free-radical emulsion polymerization in water, generally in the presence of surfactants. Emulsion polymerization is a well-known polymerization technique, and, for further details, reference may be made, for example, to the book “Latexes, Preparation, Characterisation and Applications”, edited by E. S. Daniels in the ACS Symposium Series editions (ISBN 0-8412-2305-X).

The monomers forming the latices used herein may be chosen from any of those known in the art as being capable of being polymerized in aqueous emulsion.

The monomers may, for example, be chosen from vinylaromatic compounds such as styrene, methylstyrene and divinylbenzene; diene monomers such as butadiene, dimethylbutadiene, isoprene, 1,3-hexadiene and chloroprene; C₁-C₁₀ alkyl and aryl acrylates and methacrylates such as methyl, ethyl, n-butyl, 2-ethylhexyl, t-butyl, isobornyl, phenyl and benzyl (meth)acrylates; vinyl acetate; acrylonitrile; vinyl chloride and vinylidene chloride; alkenes such as ethylene and propylene; fluorinated monomers and telomers such as fluoroalkyl (meth)acrylate and alkyl α-fluoroacrylates; acrylamide and the alkyl derivatives thereof such as N-methylacrylamide and N-isopropylacrylamide; methacrylamide; polyethylene glycol (meth)acrylate; N-vinylacetamide; N-methyl-N-vinylacetamide; N-vinylformamide; N-methyl-N-vinylformamide; N-vinyllactams comprising at least one group chosen from C₄-C₉ cyclic alkyl groups; acrylic acid; methacrylic acid; and hydroxyethyl and hydroxypropyl (meth)acrylates.

The above monomers must form, after polymerization, at least one water-dispersed polymer with a glass transition temperature of greater than 30° C. The at least one water-dispersed polymer may, for example, be chosen from, without this list being exhaustive, polystyrene, poly(vinylacetate), poly(α-methylstyrene), poly(acrylamide), poly(acrylonitrile), poly(vinyl chloride), copolymers based on styrene and C₁-C₄ alkyl (meth)acrylates, copolymers based on styrene and acrylamide, copolymers based on styrene and acrylonitrile, copolymers based on styrene and vinyl acetate, copolymers based on acrylamide and C₁-C₄ alkyl (meth)acrylates, copolymers based on acrylonitrile and C₁-C₄ alkyl (meth)acrylates, copolymers based on acrylonitrile and acrylamide, terpolymers based on styrene, acrylonitrile and acrylamide, poly(methyl methacrylate), poly(ethyl methacrylate), and styrene/butadiene, styrene/acrylic acid, styrene/vinylpyrrolidone and butadiene/acrylonitrile copolymers.

The mean size of the latex particles disclosed herein may range, for example, from 10 nm to 200 nm. Above this upper limit, it may be very difficult to immobilize the particles on keratin fibers and the desired effect, i.e., an increase in the volume of the hair, can be insufficient. The inventor has obtained satisfactory results, for example, with particles with a mean particle size ranging, for example, from 50 to 150 nm. As used herein, the term “particle size” means the maximum size that it is possible to measure between two diametrically opposite points of the particle. The techniques for measuring the mean size of a suspension of particles include direct observation by microscopy—for example, by scanning electron microscopy or atomic force microscopy—but well-known indirect techniques such as dynamic light scattering may also be used.

The non-film-forming cationic latex particles disclosed herein may, for example, be particles of spherical shape, but they may also, for example, be particles of random shape. The internal structure of the latex particles may, for example, be chosen from core-sheath type internal structures, sandwich type internal structures, and particles resulting from the aggregation of spheres (“raspberry”). The mean overall size of such aggregates of several spheres should, however, be within the limits indicated above. For further details regarding the various structures of latex, reference may be made to pages 235-238 of the above-referenced book “Latexes, Preparation, Characterisation and Applications”.

The non-film-forming nature at room temperature (about 20° C.) of the cationic latex particles used is an essential characteristic of the hair treatment compositions disclosed herein. Specifically, as explained above, the adsorbed particles should not form a polymer film on the surface of the keratin fibers, since this could result in an undesirable change in the feel of the treated hair. The non-film-forming nature at room temperature of the polymers that may be used in the compositions disclosed herein results from a glass transition temperature (T_{g}), measured by differential calorimetric analysis (DSC, Differential Scanning Calorimetry) with a temperature gradient of 10° C./minute, of greater than or equal to 30° C. Such a polymer will be, at room temperature, in a vitreous state that does not allow plastic deformation, which is essential for forming a film.

The cationic latex particles that may, for example, be used in the compositions disclosed herein may have a glass transition temperature (T_{g}), for example, greater than 40° C. and further, for example, greater than 50° C.

The cationic nature of the latices used in the compositions disclosed herein is expressed by the zeta potential (ζ), i.e., the potential difference existing between the shear plane (hydrodynamic cleavage plane) close to the surface of the particles and the center of the suspension liquid. This shear plane is in the region of the outer Helmholtz plane, i.e., between the rigid layer (Stern layer) and the diffuse layer (Gouy-Chapman layer) of ions surrounding the particles. The zeta potential is expressed in volts, the millivolt being the unit most commonly used on account of the small potential different values measured. The zeta potential is conventionally determined by electrophoresis, i.e. by measuring the speed of travel of the particles in an electric field. This speed is determined by light scattering using the Doppler effect. Such machines for measuring the zeta potential (electrophoresis+Doppler-effect laser) are sold, for example, by the company Malvern Instruments under the name Zetasizer or Zetamaster.

For further details regarding the definition of the zeta potential and its measurement, reference may be made to the book entitled “Phenomenes d'interfaces, Agents de surface” [Interface Phenomena and Surface Agents] by J. Briant, published in the Technip editions (ISBN 2.7108.0578-2), on pages 165-203 and pages 223-243, respectively.

The zeta potential value depends greatly on the measuring conditions and, for example, on the composition of the suspension medium (ionic strength, pH). All the zeta potential values disclosed herein were measured under the following standard conditions:

measuring machine: Zetamaster, Malvern company,

particle suspension comprising 50 ppm of active material (latex) in Milli-Q demineralized water,

pH=7, adjusted by adding HCl or KOH,

ionic strength of the medium set by adding 1.3 mM/l of KCl,

temperature: 25° C.

The zeta potential of the cationic latex particles used in the compositions disclosed herein may, for example, be greater than +40 mV and further, for example, may be greater than +60 mV.

Various techniques exist for introducing cationic charges into synthetic polymer lattices. For example, the cationic charges may be introduced by at least one technique chosen from:

the use of at least one cationic free-radical initiator such as azobisbutyroamidinium and 2,2′-azobis(2-amidinopropane) dihydrochloride; this technique, which results in the incorporation of cationic charges at the end of the polymer chain, may not make it possible to obtain a very high charge density at the surface of the particles and can be used in combination with other methods:

the use of at least one cationic surfactant such as dodecyltrimethylammonium bromide; this technique allows the production of high charge densities, which are, however, simply adsorbed onto the surface of the particles;

the copolymerization, in combination with the nonionic monomers listed above, of a certain fraction of water-insoluble cationic comonomers which thus become incorporated into the macromolecular chain of the polymer forming the particles; for example, the cationic comonomers may be chosen from N,N-dialkylaminoethyl and N,N-dialkylaminopropyl methacrylates optionally quaternized with at least one group chosen from C₁-C₆ alkyl groups; the synthesis of such a cationic latex by copolymerization of a nonionic monomer (styrene) and a cationic monomer (N,N-diethylaminoethyl methacrylate) is described, for example, in the article by B. Alince et al. in Journal of Applied Polymer Science, 76, 1677-1682 (2000); and

the grafting, i.e., the covalent attachment, of at least one cationic organic compound onto the surface of the already-formed latex particles. The at least one cationic organic compound may, for example, be chosen from compounds of low mass and polymers. As an example of the attachment of the at least one cationic polymer by grafting, mention may be made of the attachment of a thiol-containing polyethyleneimine to a latex whose surface has been prefunctionalized with at least one thiol group, according to the following reaction scheme:

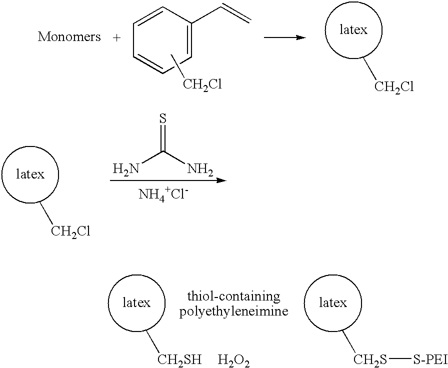

The concentration of the non-film-forming cationic latex particles in the compositions disclosed herein may, for example, range from 0.001% to 50% by weight, relative to the total weight of the composition, further, for example, from 0.05% to 5% by weight, relative to the total weight of the composition, and further, for example, ranging from 0.1% to 2% by weight, relative to the total weight of the composition.

As used herein, the expression “cosmetically acceptable medium” means a medium not comprising any ingredients that are incompatible, for toxicological reasons, with use on the human body, for example, on the skin and the integuments.

The cosmetically acceptable liquid medium may, for example, be chosen from aqueous mediums optionally comprising a certain fraction of at least one organic solvent. For example, the at least one organic solvent may be chosen from lower alkanols, for example, ethanol and isopropanol; benzyl alcohol; polyols such as glycerol; alkylene glycols and polyalkylene glycols such as propylene glycol; dipropylene glycol and butylene glycol; acetone; methyl ethyl ketone; methyl acetate; ethyl acetate; dimethoxyethane and diethoxyethane.

The hair treatment compositions disclosed herein may also comprise at least one known active principle and/or at least one known formulation adjuvant, which may, for example, be chosen from at least one of volatile and non-volatile silicones; mineral, organic and plant oils; oxyethylenated and non-oxethylenated waxes; paraffins; C₅-C₁₀ alkanes; fatty acids; fatty amides; fatty esters; fatty alcohols; reducing agents; oxidizing agents; sequestering agents; thickeners; softeners; antifoams; moisturizers; emollients; pH adjusters and regulators; sunscreens; direct dyes and oxidation dye precursors; pigments; nacreous agents; peptizers; preserving agents; surfactants; fixing polymers; plasticizers; conditioning polymers; proteins and vitamins.

On selecting these adjuvants, a person skilled in the art will take care in particular to ensure that they do not harm the intrinsic qualities of the compositions disclosed herein.

The compositions disclosed herein may be packaged in aerosol devices and may then contain at least one propellant. For example, the at least one propellant may be chosen from compressed and liquefied gases commonly employed for manufacturing aerosol compositions. Further, for example, the at least one propellant may be chosen from carbon dioxide, compressed nitrogen, dimethyl ether, hydrocarbons, and halohydrocarbons, for example, fluorohydrocarbons.

The compositions disclosed herein may, for example, be provided in a form chosen from lotions, sprays, aerosol sprays, aerosol mousses, conditioners and shampoos.

Further disclosed herein is a hair treatment process comprising

applying to wet or dry hair a composition comprising a dispersion, in a cosmetically acceptable liquid medium, of non-film-forming cationic latex particles as defined above,
optionally rinsing the hair, and
drying the hair.

The inventor has found that the non-film-forming cationic latex particles used in the treatment compositions disclosed herein can show excellent affinity for the hair, allowing them to be used not only in leave-in direct application, but also, for example, in the form of a shampoo or conditioner in rinse-out application.

The drying of the hair may be performed in the open air at room temperature, but also by supplying heat in the form of radiation or hot air.

### EXAMPLE 1

### Styling Lotion

The styling lotion A according to the present disclosure and the control lotion B below were prepared:

| | Composition A | | | |
|---|---|---|---|---|
| | (according to | | Composition B | |
| Ingredient | present disclosure) | | (control) | |
| Non-film-forming cationic | 1.0 | g of active | — | |
| latex particles* | | material | | |
| Aminomethylpropanol | qs pH 7 | | qs pH 7 | |
| Ethanol | 10 | g | 10 | g |
| Fragrance | qs | | qs | |
| Water | qs 100 | g | qs 100 | g |

*Basoplast ® 265 D, BASF

The non-film-forming cationic latex particles (Basoplast 265 D, BASF) used in composition A, according to the present disclosure, has a zeta potential of 48 mV±1 mV, a glass transition temperature, measured by DSC, of 50° C.±1° C. and a mean particle size (number-average size, determined by dynamic light scattering using an He—Ne laser and Brookhaven B19000 correlator) of 75 nm.

5 g of each of the above styling lotions (compositions A and B) were applied, respectively, to 10 heads of hair. The cosmetic characteristics of the hairstyles were evaluated by a group of 10 experts. The experts all invariably indicated that the application of the composition according to the present disclosure (composition A) comprising non-film-forming cationic latex particles gave the hair greater volume than the control composition (composition. B) and did not adversely affect the natural feel of the hair.

### EXAMPLE 2

### Styling Mousse

The two compositions C and D below were prepared:

| | Composition C | | | |
|---|---|---|---|---|
| | (according to the | | Composition D | |
| Ingredient | present disclosure) | | (control) | |
| Non-film-forming cationic | 1 | g a.m.* | — | |
| latex particles of Ex. 1 | | | | |
| Starch acetate | 5 | g a.m.* | 5 | g a.m.* |
| Polysorbate 20 | 0.1 | g a.m.* | 0.1 | g a.m.* |
| Cocamidopropylbetaine | 0.5 | g a.m.* | 0.5 | g a.m.* |
| Laureth-4 | 0.3 | g a.m.* | 0.3 | g a.m.* |
| Isobutane/butane/ | 5 | g a.m.* | 5 | g a.m.* |
| propane mixture*** | | | | |
| Preserving agent | qs | | qs | |
| Fragrance | qs | | qs | |
| Water | qs 100 | g | qs 100 | g |

*a.m. = active material
**Aeron 3.2 from the company Solvadis

5 g of each of the above styling mousses (compositions C and D above) were applied, respectively, to 10 heads of hair. The cosmetic characteristics of the hairstyles were evaluated by a group of 10 experts. The experts all invariably indicated that the application of the composition according to the present disclosure (composition C) comprising non-film-forming cationic latex particles gave the hair greater volume than the control composition (composition D), without adversely affecting the natural feel of the hair.

### EXAMPLE 3

### Shampoo

The shampoo compositions E and F having the composition below were prepared:

| | Composition E | | | |
|---|---|---|---|---|
| | (according to the | | Composition F | |
| Ingredient | present disclosure) | | (control) | |
| Non-film-forming cationic | 1 | g a.m.^{b)} | — | |
| latex particles of Ex. 1 | | | | |
| Sodium Laureth sulfate^{a)} | 20 | g a.m.^{b)} | 20 | g a.m.^{b)} |
| Cocobetaine^{a)} | 4 g | a.m.^{b)} | 4 | g a.m.^{b)} |
| Cocamide MIPA^{a)} | 2 | g a.m.^{b)} | 2 | g a.m.^{b)} |
| Sodium Cetearyl Sulfate^{a)} | 0.8 | g a.m.^{b)} | 0.8 | g a.m.^{b)} |
| Carbomer | 0.2 | g a.m.^{b)} | 0.2 | g a.m.^{b)} |
| Dimethicone^{a)} | 2 | g a.m.^{b)} | 2 | g a.m.^{b)} |
| Preserving agent | qs | | qs | |
| Fragrance | qs | | qs | |
| Water | qs 100 | g | qs 100 | g |

^{a)}CTFA Cosmetic Ingredient Handbook name
^{b)}a.m. = active material

5 g of each of the shampoo compositions E and F were applied, respectively, to 10 heads of natural Caucasian chestnut-brown hair 20 cm long, and, after an action time of 2 minutes, the heads of hair were rinsed with water and dried using a hair drier. A group of 10 experts indicated that, in all cases, the application of the composition according to the present disclosure (composition E) comprising non-film-forming cationic latex particles gave the hair more volume than the control composition (composition F) without adversely affecting the natural feel of the hair.

This last example shows that the compositions disclosed herein are effective in terms of increasing the volume of the hair even in rinse-out application mode.

What is claimed is:

1. A hair treatment composition comprising a dispersion, in a cosmetically acceptable liquid medium, of non-film forming cationic latex particles
formed from at least one synthetic polymer, obtained by free-radical polymerization, with a glass transition temperature (T_{g}) of greater than or equal to 30° C.,
having a mean particle size ranging from 10 nm to 200 nm, and
having a zeta surface potential (ζ) of greater than +20 mV.

2. The composition according to claim 1, wherein the mean size of the non-film-forming cationic latex particles ranges from 50 to 150 nm.

3. The composition according to claim 1, wherein the glass transition temperature (T_{g}) of the at least one synthetic polymer forming the non-film-forming cationic latex particles is greater than 40° C.

4. The composition according to claim 1, wherein the glass transition temperature (T_{g}) of the at least one synthetic polymer forming the non-film-forming cationic latex particles is greater than 50° C.

5. The composition according to claim 1, wherein the at least one synthetic polymer forming the non-film-forming cationic latex particles is chosen from homopolymers and copolymers based on vinylaromatic monomers; diene monomers; C₁-C₁₀ alkyl and aryl acrylates and methacrylates; vinyl acetate; acrylonitrile; vinyl chloride; vinylidene chloride; alkenes; fluorinated monomers and telomers; acrylamide and the alkyl derivatives thereof; methacrylamide; polyethylene glycol (meth)acrylate; N-vinylacetamide; N-methyl-N-vinylacetamide; N-vinylformamide; N-methyl-N-vinylformamide; N-vinyllactams comprising at least one group chosen from C₄-C₉ cyclic alkyl groups; acrylic acid; methacrylic acid; and hydroxyethyl and hydroxypropyl (meth)acrylate.

6. The composition according to claim 1, wherein the zeta potential (ζ) of the non-film-forming cationic latex particles is greater than +40 mV.

7. The composition according to claim 6, wherein the zeta potential (ζ) of the non-film-forming cationic latex particles is greater than +60 mV.

8. The composition according to claim 1, wherein the cationic charge of the latex particles is introduced by at least one technique chosen from:
using at least one cationic initiator,
using at least one cationic surfactant during emulsion polymerization,
copolymerization of cationic comonomers, and
covalent grafting of at least one cationic organic compound onto the surface of the non-film-forming cationic latex particles.

9. The composition according to claim 8, wherein the at least one cationic initiator is azobisbutyroamidinium.

10. The composition according to claim 8, wherein the at least one cationic surfactant is dodecyltrimethylammonium bromide.

11. The composition according to claim 8, wherein the cationic monomers are chosen from N,N-dialkylaminoethyl and N,N-dialkylaminopropyl methacrylates optionally quaternized with at least one group chosen from C₁-C₆ alkyl groups.

12. The composition according to claim 8, wherein the at least one organic compound grafted onto the surface of the non-film-forming cationic latex particles is a thiol-containing polyethyleneimine (PEI-SH).

13. The composition according to claim 1, wherein the non-film-forming cationic latex particles are present in an amount ranging from 0.001% to 50% by weight, relative to the total weight of the composition.

14. The composition according to claim 13, wherein the non-film-forming cationic latex particles are present in an amount ranging from 0.05% to 5% by weight, relative to the total weight of the composition.

15. The composition according to claim 14, wherein the non-film-forming cationic latex particles are present in an amount ranging from 0.1% to 2% by weight, relative to the total weight of the composition.

16. The composition according to claim 1, wherein the composition is provided in a form chosen from lotions, sprays, aerosol sprays, aerosol mousses, conditioners and shampoos.

17. A hair treatment process comprising:
applying to wet or dry hair a composition comprising a dispersion, in a cosmetically acceptable liquid medium, of non-film forming cationic latex particles
formed from at least one synthetic polymer, obtained by free-radical polymerization, with a glass transition temperature (T_{g}) of greater than or equal to 30° C.,
having a mean particle size ranging from 10 nm to 200 nm, and
having a zeta surface potential (ζ) of greater than +20 mV;
optionally, rinsing the hair; and
drying the hair.

18. A method for increasing the volume of hair comprising applying to the hair a composition comprising a dispersion, in a cosmetically acceptable liquid medium, of non-film-forming cationic latex particles
formed from at least one synthetic polymer, obtained by free-radical polymerization, with a glass transition temperature (T_{g}) of greater than or equal to 30° C.,
having a mean particle size ranging from 10 nm to 200 nm, and
having a zeta surface potential (ζ) of greater than +20 mV; wherein the non-film-forming cationic latex particles are present in a sufficient concentration for increasing the volume of the hair.
